(19) United States
(12) Patent Application Publication
(10) Pub. No.: US 2018/0153618 A1
(43) Pub. Date: Jun. 7, 2018

(54) ELECTROMAGNETIC THERMOTHERAPY ESTIMATION SYSTEM AND ELECTROMAGNETIC THERMOTHERAPY ESTIMATION METHOD

(71) Applicant: Metal Industries Research & Development Centre, Kaohsiung (TW)

(72) Inventors: Chien-Chang Chen, Kaohsiung City (TW); Ying-Cherng Lu, Kaohsiung City (TW); Chiu-Feng Lin, Pingtung County (TW); Chun-Sen Wu, Kaohsiung City (TW)

(73) Assignee: Metal Industries Research & Development Centre, Kaohsiung (TW)

(21) Appl. No.: 15/371,216

(22) Filed: Dec. 7, 2016

(51) Int. Cl. A61B 34/10; A61B 34/00; A61B 5/01; A61B 5/00

(52) U.S. Cl. CPC A61B 34/10; A61B 34/25; A61B 2018/00577; A61B 5/4848; A61B 5/01

(57) ABSTRACT

An electromagnetic thermotherapy estimation system adapted to estimate an ablation result of a biological tissue ablated by a needle induced by an electromagnetic coil is provided. The electromagnetic thermotherapy estimation system includes an input device and a processing device. The input device is configured to receive a plurality of setting parameters. The setting parameters include a needle tip depth and a current magnitude. The processing device is electrically connected to the input device, and has a database configured to store an ablation range estimation model and a temperature envelope estimation model. The processing device is configured to calculate ablation range data and temperature envelope data corresponding to the setting parameters according to the ablation range estimation model, the temperature envelope estimation model and the setting parameters. In addition, an electromagnetic thermotherapy estimation method is also provided.

### BACKGROUND OF THE INVENTION

### 1. Field of the Invention

The invention relates to an estimation technique, and more particularly, to an electromagnetic thermotherapy estimation system and an electromagnetic thermotherapy estimation method.

### 2. Description of Related Art

Cancer (also known as tumor) is one of major human diseases ranked as the top three of statistical death factors in many countries. Thus, not only is cancer treatment an urgent medical need in those countries, research and development on various medical equipments for cancer-related treatment has also becomes very important in the related field. In particular, a thermotherapy surgery on tumor is currently one of main cancer treatment techniques. For example, the main cancer treatment techniques, such as RFA (Radio Frequency Ablation) or MWA (Microwave Ablation) in tumor ablation surgery, are now applicable in local tumor treatment.

On the other hand, a thermotherapy system based on EMA (Electromagnetic Ablation) is also available. However, because the current electromagnetic thermotherapy system is still lack of an estimation technique for post-ablation temperature area range, medical professionals are unable to clearly learn about ablation conditions at the site of treatment for patients so proper commands or operations cannot be promptly given to the thermotherapy system. For instance, said ablation conditions can involve an amount of current to go through a magnetic field generator, a length of an ablation time, a needle tip depth for inserting a needle, whether an ablation range on a biological tissue to be ablated meets the criteria, and the like. In other words, if physical characteristics of the biological tissue an operation time on which cannot be precisely handled in the practice, a normal tissue may be inadvertently removed since a diameter of high temperature area generated by energy-based surgical instruments may become overly large. Alternatively, a complete ablation result cannot be effectively achieved if the diameter of the high temperature area is overly small.

Accordingly, it is required to ensure that the electromagnetic ablation can provide a safe treatment range in order to improve a treatment quality as well as surgical safety and accuracy for patients. Therefore, finding a way to effectively estimate a temperature area range and a temperature variation of the needle over time during the electromagnetic ablation is one of important issues to be addressed. In view of the above, several embodiments of the invention are provided as follows.

### SUMMARY OF THE INVENTION

The invention is directed to an electromagnetic thermotherapy estimation system and an electromagnetic thermotherapy estimation method, which are capable of correspondingly calculating ablation range data and temperature envelope data according to inputted setting parameters in order to effectively estimate preoperative estimation information regarding ablation range and temperature envelope.

The electromagnetic thermotherapy estimation system of the invention is adapted to estimate an ablation result of a biological tissue ablated by a needle induced by an electromagnetic coil. The electromagnetic thermotherapy estimation system includes an input device and a processing device. The input device is configured to receive a plurality of setting parameters. The setting parameters include a needle tip depth and a current magnitude. The processing device is electrically connected to the input device, and has a database configured to store an ablation range estimation model and a temperature envelope estimation model. The processing device is configured to calculate ablation range data and temperature envelope data corresponding to the setting parameters according to the ablation range estimation model, the temperature envelope estimation model, and the setting parameters.

In one embodiment of the invention, the database is further configured to store a plurality of first thermal physical parameters of the biological tissue and store a plurality of second thermal physical parameters of the needle and the electromagnetic coil. The processing device is configured to perform an electromagnetic simulation analysis and a heat transfer simulation analysis according to the first thermal physical parameters and the second thermal physical parameters so as to create the ablation range estimation model and the temperature envelope estimation model.

In one embodiment of the invention, the first thermal physical parameters include a density, a specific heat capacity value, a thermal conductivity and an electrical conductivity of the biological tissue.

In one embodiment of the invention, the second thermal physical parameters include a density, a specific heat capacity value, a thermal conductivity and an electrical conductivity of the needle and a surface current density, a radiant flux and an electrical conductivity of the electromagnetic coil.

In one embodiment of the invention, the setting parameters further include an ablation time. The processing device calculates temperature variation data of the needle according to a temperature characteristic equation and the setting parameters.

In one embodiment of the invention, the temperature characteristic equation further includes a plurality of parameter values. The parameter values include a needle tip temperature, a needle tail temperature, a nonlinear regression parameter value, a thermotherapy execution time and a time-varying parameter value of a temperature index.

In one embodiment of the invention, the processing device performs a plurality of sampling ablation operations having different ablation times, different needle tip depths and different current magnitudes on another biological tissue in advance by the needle, and analyzes a needle tip temperature and a needle tail temperature of the needle in each of the sampling ablation operations through a nonlinear regression analysis so as to create the temperature characteristic equation.

In one embodiment of the invention, the electromagnetic thermotherapy estimation system further includes a driving circuit, a current sensor and a temperature sensor. The driving circuit is electrically connected to the electromagnetic coil. The driving circuit is configured to generate an alternating magnetic field through the electromagnetic coil so as to induce the needle. The current sensor is electrically connected to the driving circuit. The current sensor is configured to sense a plurality of current values provided by the driving circuit respectively corresponding to a plurality of sampling ablation results. The temperature sensor is electrically connected to the needle. The temperature sensor is configured to sense a plurality of temperature values of the needle respectively corresponding to the sampling ablation results. The processing device creates the temperature characteristic equation through the nonlinear regression analysis according to the current values and the temperature values corresponding to the different ablation times and the different needle tip depths.

In one embodiment of the invention, the display device is electrically connected to the processing device. The display device is configured to display an ablation range image and at least one temperature envelope corresponding to the setting parameters according to the ablation range data and the temperature envelope data. The ablation range image comprises marking a maximum ablation width.

In one embodiment of the invention, the display device is further configured to display a temperature variation curve of the needle according to the temperature variation data.

The electromagnetic thermotherapy estimation method of the invention is adapted to estimate an ablation result of a biological tissue ablated by a needle induced by an electromagnetic coil. The electromagnetic thermotherapy estimation method includes the following step. A plurality of setting parameters is received, where the setting parameters include a needle tip depth and a current magnitude. Ablation range data and temperature envelope data corresponding to the needle tip depth and the current magnitude are calculated according to an ablation range estimation model, a temperature envelope estimation model and the setting parameters.

In one embodiment of the invention, the electromagnetic thermotherapy estimation method further includes the following step. An electromagnetic simulation analysis and a heat transfer simulation analysis are performed according to a plurality of first thermal physical parameters of the biological tissue and a plurality of second thermal physical parameters of the needle and the electromagnetic coil so as to create the ablation range estimation model and the temperature envelope estimation model.

In one embodiment of the invention, the first thermal physical parameters include a density, a specific heat capacity value, a thermal conductivity and an electrical conductivity of the biological tissue.

In one embodiment of the invention, the second thermal physical parameters include a density, a specific heat capacity value, a thermal conductivity and an electrical conductivity of the needle and a surface current density, a radiant flux and an electrical conductivity of the electromagnetic coil.

In one embodiment of the invention, the setting parameters further include an ablation time. The electromagnetic thermotherapy estimation method further includes the following step. Temperature variation data of the needle is calculated according to a temperature characteristic equation and the setting parameters.

In one embodiment of the invention, the electromagnetic thermotherapy estimation method further includes the following step. A plurality of sampling ablation operations having different ablation times, different needle tip depths and different current magnitudes are performed on another biological tissue in advance by the needle. A needle tip temperature and a needle tail temperature of the needle in each of the sampling ablation operations are analyzed through a nonlinear regression analysis so as to create the temperature characteristic equation.

In one embodiment of the invention, the step of creating the temperature characteristic equation includes the following step. An alternating magnetic field is generated by driving the electromagnetic coil through a driving circuit so as to induce the needle. A plurality of current values provided by the driving circuit respectively corresponding to a plurality of sampling ablation results are sensed through a current sensor. A plurality of temperature values of the needle respectively corresponding to the sampling ablation results are sensed through a temperature sensor. The temperature characteristic equation is created through the nonlinear regression analysis according to the current values and the temperature values corresponding to the different ablation times and the different needle tip depths.

In one embodiment of the invention, the electromagnetic thermotherapy estimation method further includes the following step. An ablation range image and at least one temperature envelope corresponding to the setting parameters are displayed through a display device according to the ablation range data and the temperature envelope data. The ablation range image comprises marking a maximum ablation width.

In one embodiment of the invention, the electromagnetic thermotherapy estimation method further includes the following step. A temperature variation curve corresponding to the setting parameters is displayed through the display device according to the temperature variation data.

Based on the above, the electromagnetic thermotherapy estimation system and the electromagnetic thermotherapy estimation method according to the embodiments of the invention can be used to calculate the ablation range data and the temperature envelope data corresponding to the setting parameters according to the ablation range estimation model and the temperature envelope estimation model created in advance in the database and the inputted setting parameters. In this way, the electromagnetic thermotherapy estimation system according to the embodiments of the invention can effectively estimate the ablation range and the temperature envelope caused by ablating the biological tissue by the needle induced by the electromagnetic coil according to the setting parameters provided by medical professionals. As a result, the electromagnetic thermotherapy estimation system according to the embodiments of the invention can effectively improve ablation safety and thermotherapy quality by effectively providing accurate preoperative estimation information.

To make the above features and advantages of the invention more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a block diagram illustrating an electromagnetic thermotherapy estimation system in an embodiment of the invention.

FIG. 2 is a schematic structural view of a needle in an embodiment of the invention.

FIG. 3 is a block diagram illustrating an ablation operation in electromagnetic thermotherapy in an embodiment of the invention.

FIGS. 4A to 4C are schematic diagrams illustrating distribution of temperature envelopes corresponding to different current magnitudes in an embodiment of the invention.

FIG. 5 is a block diagram illustrating an electromagnetic thermotherapy estimation system in another embodiment of the invention.

FIG. 6 is a flowchart illustrating steps for inducing the needle by the electromagnetic coil in an embodiment of the invention.

FIG. 7 is a schematic diagram illustrating a temperature curve in an embodiment of the invention.

FIG. 8 is a flowchart illustrating steps of an electromagnetic thermotherapy estimation method in an embodiment of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

Embodiments are provided below to describe the invention in detail, though the invention is not limited to the provided embodiments, and the provided embodiments can be suitably combined. The term “electrically connected” used in this specification (including claims) of the present application may refer to any direct or indirect connection means. For example, “a first device is electrically connected to a second device” should be interpreted as “the first device is directly connected to the second device” or “the first device is indirectly connected to the second device through other devices or connection means”.

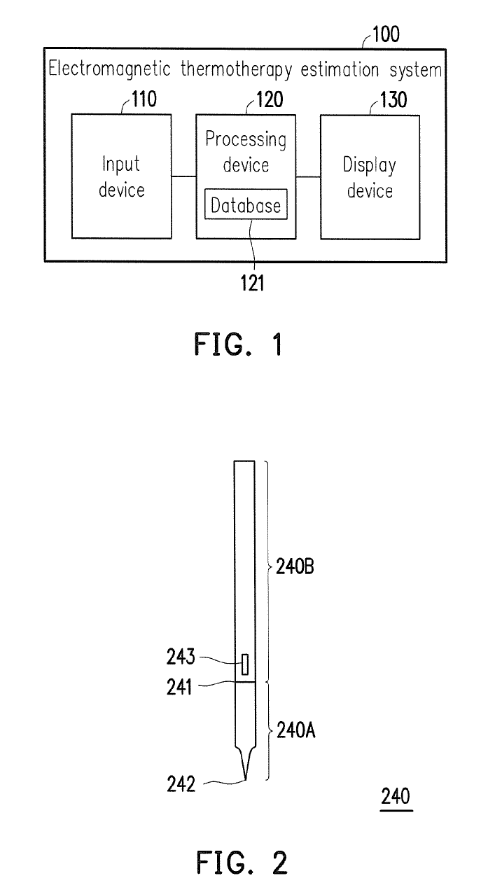

FIG. 1 is a block diagram illustrating an electromagnetic thermotherapy estimation system in an embodiment of the invention. Referring to FIG. 1, an electromagnetic thermotherapy estimation system 100 includes an input device 110, a processing device 120 and a display device 130. In the present embodiment, the input device 110 is electrically connected to the processing device 120. The input device 110 is configured to receive a plurality of setting parameters inputted from the outside and provide a plurality of setting parameters to the processing device 120. The processing device 120 has a database 121, which is configured to store a plurality of estimation models. In the present embodiment, the processing device 120 can calculate for data corresponding to the setting parameters according to said estimation models and said setting parameters in the database 121, and display an estimation result through the display device 130.

In the present embodiment, the input device 110 may be, for example, a physical component, such as physical keyboard, mouse, button or touchpad, and the like. Alternatively, the input device 110 may also be, for example, a software component such as an input interface. For instance, the display device 130 may be, for example, a display with touch functions. The display device 130 can display image information of the input interface so medical professionals can input the setting parameters by touching on the display device 130. Alternatively, medical professionals may also input the setting parameters by using an additional physical keyboard, but the invention is not limited to the above. In this regard, enough teaching, suggestions and implementations for the input device 110 and the display device 130 of the present embodiment may be obtained according to the common knowledge in the field, which are not repeated hereinafter.

In the present embodiment, the processing device 120 may be, for example, a central processing unit (CPU) composed of single-core or multi-core, a microprocessor for general purpose or special purpose, a digital signal processor (DSP), a programmable controller, an application specific integrated circuits (ASIC), a programmable logic device (PLD) or other similar devices, or a combination of the above devices, which are capable of the electromagnetic thermotherapy estimation method in each embodiment of the invention. Also, the processing device 120 may further include a memory component. The memory component may be, for example, a random access memory (RAM), a read-only memory (ROM) or a flash memory and the like, which may be at least used to store the database described in each embodiment of the invention. Further, the database can store various parameter data and the estimation models described in each embodiment of the invention.

Specifically, the electromagnetic thermotherapy estimation system 100 of the present embodiment is used to estimate an ablation result of a biological tissue ablated by a needle induced by an electromagnetic coil. In the present embodiment, the processing device 120 can store an ablation range estimation model and a temperature envelope estimation model in advance. Medical professionals can input the setting parameters through the input device 110, where the setting parameters can include a needle tip depth and a current magnitude. By doing so, the processing device 120 can calculate ablation range data and temperature envelope data corresponding to the setting parameters according to the ablation range estimation model, the temperature envelope estimation model, and the setting parameters. Further, the display device 130 can display an image frame having the ablation range and a temperature envelope corresponding to the setting parameters according to the ablation range data and the temperature envelope data. It should be noted that, in the present embodiment, the needle tip depth refers to a distance from the electromagnetic coil to a needle tip of the needle. The current magnitude refers to a current value for driving the electromagnetic coil so the electromagnetic coil can generate an alternating magnetic field to induce the needle.

In the present embodiment, the database 121 of the processor 120 can be used to store a plurality of first thermal physical parameters of the biological tissue and store a plurality of second thermal physical parameters of the needle and the electromagnetic coil. In the present embodiment, the biological tissue may be, for example, in vitro tissue or living tissue. Further, the biological tissue may be, for example, tissue parts of various organs in human or animal body, such as thyroid or liver tissue, which are not particularly limited by the invention. The first thermal physical parameters and the second thermal physical parameters may be obtained in advance from medical journal literatures or clinical trials, and built into the database 121. In the present embodiment, the first thermal physical parameters include, for example, a density, a specific heat capacity value, a thermal conductivity and an electrical conductivity of the biological tissue. The second thermal physical parameters include, for example, a density, a specific heat capacity value, a thermal conductivity and an electrical conductivity of the needle and a surface current density, a radiant flux and an electrical conductivity of the electromagnetic coil, but the invention is not limited to the above.

In the present embodiment, the processing device 120 can create geometric structure models of the electromagnetic coil, the needle and the biological tissue, and perform a composite physical quantity analysis with a finite element method to create modules for an electromagnetic analysis and a heat transfer analysis separately according to the first thermal physical parameters and the second thermal physical parameters. In other words, before the estimation operation is performed by the electromagnetic thermotherapy estimation system 100, the processing device 120 can create the ablation range estimation model and the temperature envelope estimation model in advance according to the first thermal physical parameters and the second thermal physical parameters. In this way, during the estimation operation performed by the electromagnetic thermotherapy estimation system 100, the processing device 120 can directly use the ablation range estimation model, the temperature envelope estimation model and the setting parameters pre-stored in the database 121 to obtain the corresponding ablation range data and the temperature envelope data so the image information having the ablation range and the temperature envelope may be displayed by the display device 130. In addition, enough teaching, suggestions and implementations related to actual calculation of the finite element method described above may be obtained according to the common knowledge in the field, which are not repeated hereinafter.

FIG. 2 is a schematic structural view of a needle in an embodiment of the invention. The needle described in each embodiment of the invention may be, for example, a needle 240 depicted in FIG. 2. Referring to FIG. 2, the needle 240 can include a magnetic induction area 240A and a non magnetic induction area 240B. One end of the magnetic induction area 240A is a needle tail 241, whereas the other end is a needle tip 242. Among them, the magnetic induction area 240A may be, for example, a solid medical grade metal made of materials like silver, platinum, stainless steel, titanium or titanium alloy, and the non magnetic induction area 240B may be, for example, made of materials like a medical grade ceramic. Further, one end of the non magnetic induction area 240B connecting to the magnetic induction area 240A includes a thermocouple 243. The thermocouple 243 may be used to sense a temperature value of the needle tail 241 of the magnetic induction area 240A. In the present embodiment, a temperature value of the needle tip 242 may be derived from the sensed temperature value of the needle tail 241 according to the material characteristics and heat transfer characteristics of the needle 240. Nonetheless, the needle 240 depicted in FIG. 2 is merely an exemplary embodiment for describing a structural relation and a temperature sensing method of the needle in each embodiment of the invention, that is, sizes and relative positions of the magnetic induction area 240A and the non magnetic induction area 240B are not limited to the above. Related specification and volume size of the needle may be designed based on different medical conditions or ablation targets.

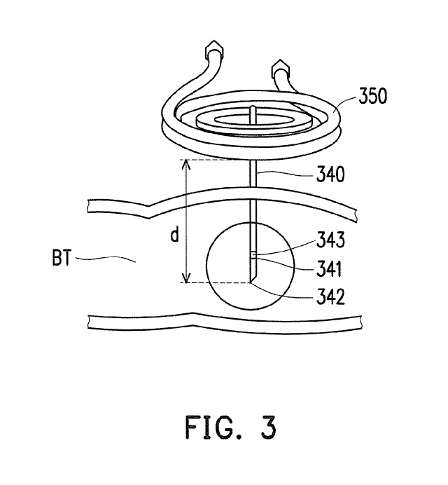

FIG. 3 is a block diagram illustrating an ablation operation in electromagnetic thermotherapy in an embodiment of the invention. Referring to FIG. 3, FIG. 3 is illustrated as one example of the ablation operation described in each embodiment of the invention. In the present embodiment, a needle 340 may be inserted to a biological tissue BT for a needle tip depth d to ablate a specific portion within the biological tissue BT, where the needle tip depth d is a distance from a needle tip 342 of the needle 340 to an electromagnetic coil 350. In the present embodiment, the needle 340 can be heated in a manner of electromagnetic induction by the electromagnetic coil 350 to rise the temperature of the magnetic induction area between the needle tail 341 and the needle tip 342. In other words, because the needle 340 has the magnetic induction area and the non magnetic induction area (e.g., as shown in FIG. 2), the needle 340 only ablates the specific portion in the biological tissue BT without damaging other portions after the needle 340 is inserted to the biological tissue BT and heated. In addition, the needle 340 can include a thermocouple 343 for sensing a temperature value of the needle to learn of a heating degree of the needle 340.

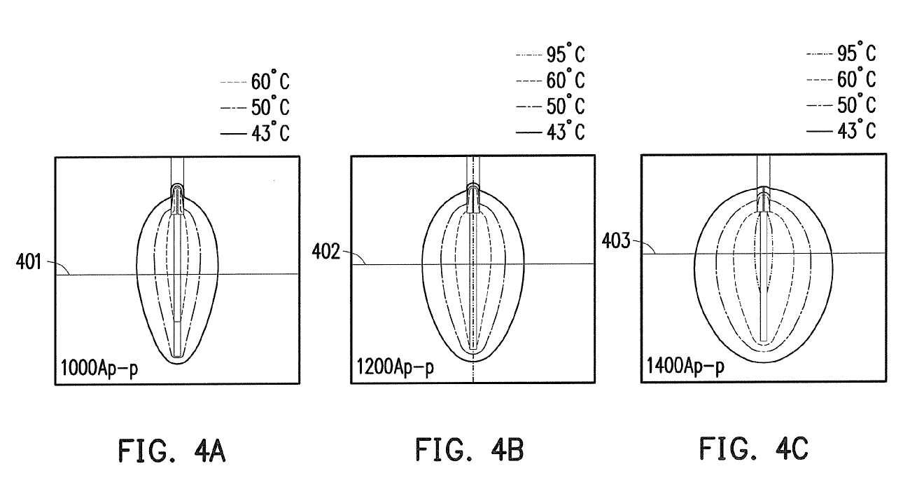

FIGS. 4A to 4C are schematic diagrams illustrating distribution of temperature envelopes corresponding to different current magnitudes in an embodiment of the invention. Referring to FIG. 3 and FIGS. 4A to 4C together, the temperature envelopes estimated by the electromagnetic thermotherapy estimation system in each embodiment of the invention are as shown by FIGS. 4A to 4C, for example. It should be noted that, distribution of the temperature envelopes shown in FIGS. 4A to 4C are schematic diagrams based on the same ablation time but different current magnitudes, where the different current magnitudes correspond to different ablation ranges, respectively. Specifically, FIG. 4A is distribution of the temperature envelopes generated from the ablation operation of the needle 340 in the biological tissue BT when the current value of the electromagnetic coil 350 is 1000 amps (Ap-p). FIG. 4B is distribution of the temperature envelopes generated from the ablation operation of the needle 340 in the biological tissue BT when the current value of the electromagnetic coil 350 is 1200 amps. FIG. 4C is distribution of the temperature envelopes generated from the ablation operation of the needle 340 in the biological tissue BT when the current value of the electromagnetic coil 350 is 1400 amps.

As shown in FIG. 4A, when the current value is 1000 amps, the needle 340 generates the temperature envelopes of 43° C., 50° C. and 60° C., and a maximum ablation width of the temperature envelope of 43° C. may be, for example, a distance between two intersected points of the temperature envelope and a reference line 401. As shown in FIG. 4B, when the current value is 1200 amps, the needle 340 generates the temperature envelopes of 43° C., 50° C., 60° C. and 95° C., and a maximum ablation width of the temperature envelope of 43° C. may be, for example, a distance between two intersected points of the temperature envelope and a reference line 402. As shown in FIG. 4C, when the current value is 1400 amps, the needle 340 generates the temperature envelopes of 43° C., 50° C., 60° C. and 95° C., and a maximum ablation width of the temperature envelope of 43° C. may be, for example, a distance between two intersected points of the temperature envelope and a reference line 403. In other words, when the current value for driving the electromagnetic coil 350 is greater, the temperature on the needle 340 becomes higher so the maximum ablation width of the temperature envelope is also greater. It should be noted that, the temperature values of 50° C. and 60° C. may, for example, represent an effective temperature for tumor carbonization, and the temperature value of 43° C. may, for example, represent a threshold preventing the biological tissue from damages. In this way, before the ablation begins, medical professionals can learn about the possible ablation results and a size of the ablation range according to distribution of the temperature envelopes presented in FIG. 4A to 4C as estimated by the electromagnetic thermotherapy estimation system, so as to effectively handle ablation-related settings.

Incidentally, the temperature envelopes presented in FIGS. 4A to 4C can further present an estimated range of the ablation range by using a color variation which gradually changes according to different temperature values. For example, in a direction extending outwardly from the needle, said color variation can gradually change from red to blue so as to present a change from a higher temperature to a lower temperature, but the invention is not limited thereto. In addition, the current magnitudes and the temperature values of the envelopes are not limited only to be various values shown in FIGS. 4A to 4C, and instead, each of the current magnitudes and the temperature values of the envelopes may be set depending on different medical needs.

The following refers to FIG. 1 again. Here, medical professionals can input the setting parameters including a specific needle tip depth and a specific current magnitude by using the input device 110 of the electromagnetic thermotherapy estimation system 100 so as to obtain the corresponding ablation range data and the temperature envelope data through the estimation performed by the processing device 120. Further, the display device 130 can display distribution of the temperature envelopes and ablation range information in, for example, one of FIGS. 4A to 4C according to the ablation range data and the temperature envelope data. In this way, medical professionals can effectively estimate the ablation result corresponding to the setting parameters by using the electromagnetic thermotherapy estimation system 100.

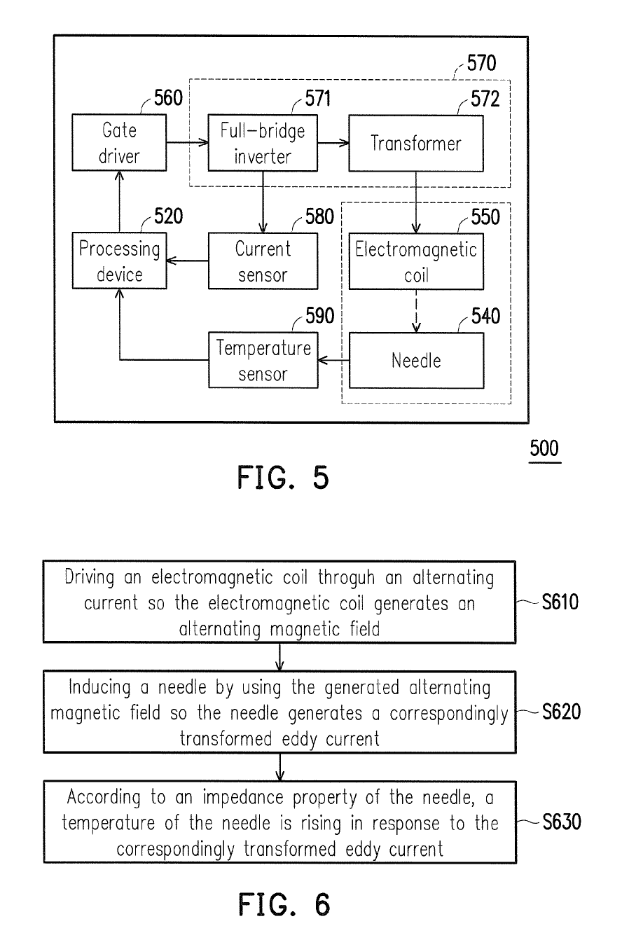

FIG. 5 is a block diagram illustrating an electromagnetic thermotherapy estimation system in another embodiment of the invention. Referring to FIG. 5, an electromagnetic thermotherapy estimation system 500 includes a processing device 520, a needle 540, an electromagnetic coil 550, a gate driver 560, a driving circuit 570, a current sensor 580 and a temperature sensor 590. In the present embodiment, the processing device 520 can control the driving circuit 570 by using the gate driver 560. The driving circuit 570 includes a full-bridge inverter 571 and a transformer 572. In the present embodiment, the electromagnetic coil 550 may be driven by the transformer 572 of the driving circuit 570 so the electromagnetic coil 550 can heat up the needle 540 in the manner of electromagnetic induction. In the present embodiment, the current sensor 580 is electrically connected to the full-bridge inverter 571. The processing device 520 can sense a current value of the driving circuit 570 by using the current sensor 580. In addition, the processing device 520 can also sense a temperature value of the needle 540 by using the temperature sensor 590. Among them, the temperature sensor 590 may be electrically connected to, for example, the thermocouple shown in FIG. 2 and FIG. 3. Accordingly, the electromagnetic thermotherapy estimation system 500 of the present embodiment can obtain the related current values and temperature values during the ablation operation. It should be noted that, in addition to the device characteristics shown in FIG. 1, the electromagnetic thermotherapy estimation system in each embodiment of the invention can further include each device characteristic for heating up the electromagnetic coil as shown in FIG. 5.

Specifically, FIG. 6 is a flowchart illustrating steps for inducing the needle by the electromagnetic coil in an embodiment of the invention. Referring to FIG. 5 and FIG. 6, the ablation operation described in each embodiment of the invention may be performed by the following steps. In step S610, the electromagnetic thermotherapy estimation system 500 provides an alternating current to the electromagnetic coil 550 so the electromagnetic coil 550 correspondingly generates an alternating magnetic field. Next, due to cutting-of-flux acted on the needle 540, the magnetic induction area of the needle correspondingly generates an electromotive force for resisting external change of flux. In other words, in step S620, based on Faraday's Law and Lenz's Law, the electromagnetic thermotherapy estimation system 500 induces the needle 540 by using the generated alternating magnetic field so the needle 540 generates a correspondingly transformed eddy current (Iₑ). Also, based on Joule's Laws, since the needle 540 has an equivalent resistance (R), a power loss (Iₑ²R) occurs on the eddy current generated by induction on the needle 540 thereby causing the temperature of the needle 540 to rise. As such, in step S630, according to an impedance property of the needle 540, the temperature of the needle 540 is rising in response to the correspondingly transformed eddy current. In this way, the needle 540 can perform the ablation operation on the biological tissue around the magnetic induction area (as shown in FIG. 3).

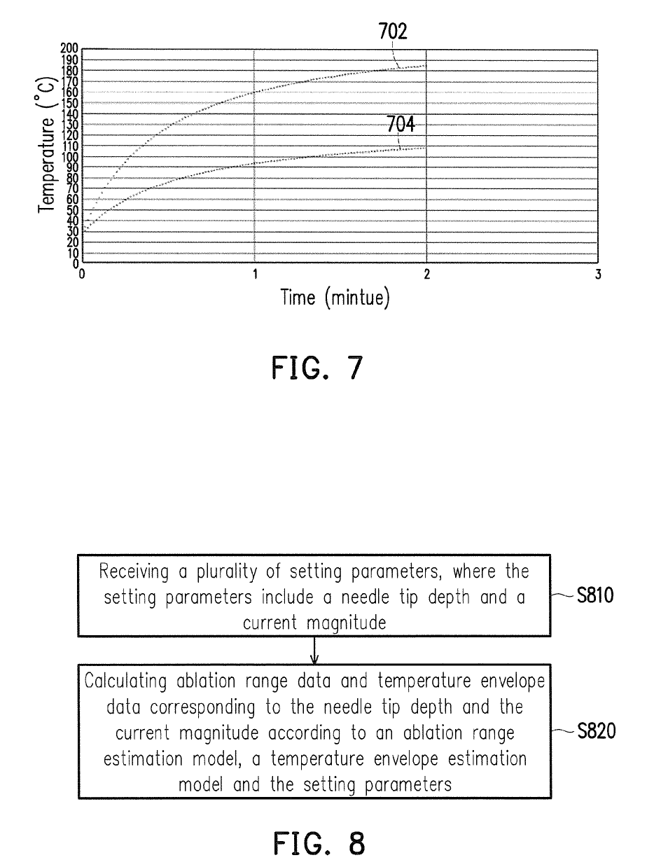

FIG. 7 is a schematic diagram illustrating a temperature curve in an embodiment of the invention. The followings refer to FIG. 1 and FIG. 7 together. In the present embodiment, the setting parameters received by the input device 110 can further include an ablation time. The processing device 120 can further store a temperature characteristic equation in the database 121. Also, the processing device 120 of the present embodiment can calculate temperature variation data of the needle according to the temperature characteristic equation and the setting parameters. It should be noted that, the temperature variation data refers a temperature variation curve generated when the electromagnetic coil heats up the needle to ablate the biological tissue (e.g., temperature variation at the magnetic induction area 240A shown in FIG. 2). In the present embodiment, the display device 130 can display a temperature variation curve 702 of a needle tip temperature and a temperature variation curve 704 of a needle tail temperature for the needle estimated by the processing device 120. Accordingly, medical professionals can effectively take a length of the ablation time into consideration according to the estimated temperature variation curves 702 and 704. Moreover, medical professionals can also correspondingly adjust the current magnitude for driving the electromagnetic coil, the length of the ablation time and the needle tip depth of the needle for inserting the biological tissue according to aforesaid temperature information, so as to effectively control the ablation result of the biological tissue. Incidentally, said ablation time refers to a time length between the beginning of driving the electromagnetic coil and the end of driving the electromagnetic coil.

In the present embodiment, the processing device 120 can obtain a plurality of sampling ablation results through a plurality of sampling ablation operations so as to create the temperature characteristic equation in advance. Specifically, medical professionals can perform ablation experiments (e.g., the ablation operation described in embodiments of FIG. 5 and FIG. 6) with different ablation times, different current magnitudes and different needle tip depths for a plurality of additional biological tissues by using the needle to obtain the corresponding temperature values so that the processing device 120 can obtain the sampling ablation results. Further, after the sampling ablation results corresponding to the different ablation times are obtained by the processing device 120, the processing device 120 can perform a regression analysis for the temperature values and the current values corresponding to the different ablation times and the different needle tip depths through a nonlinear regression analysis so as to obtain parameter values related to the temperature characteristic equation. In addition, enough teaching, suggestions and implementations related to actual calculation of the nonlinear regression analysis described above may be obtained according to the common knowledge in the field, which are not repeated hereinafter.

In the present embodiment, the temperature characteristic equation may be expressed by Equation (1) below.

(T₁−T₂)=K₁*(t^{K}^{₂}) (1)

In Equation (1), T₁ is the needle tip temperature of the magnetic induction area, T₂ is the needle tail of the magnetic induction area, K₁ is a nonlinear regression parameter value for the specific current value and the needle top depth, K₂ is a time-varying parameter of a temperature index and t is a thermotherapy execution time. For instance, after going through the calculation with the nonlinear regression analysis, the nonlinear regression parameter value K₁ may, for example, fall between 1 and 100 and the time-varying parameter value K₂ may, for example, fall between 0.1 and 0.5, but the invention is not limited thereto. Ranges of the nonlinear regression parameter value K₁ and the time-varying parameter value K₂ may be correspondingly adjusted based on different needle specifications, different electromagnetic coil specifications or different biological tissue characteristics.

In other words, in addition to the estimation on the ablation range and the range of the temperature envelopes caused by ablating the biological tissue by the needle, the electromagnetic thermotherapy estimation system 100 of the present embodiment can also estimate the temperature variation on the needle heated by the electromagnetic coil. In this way, when medical professionals use the electromagnetic thermotherapy estimation system 100 to estimate the ablation operation, medical professionals can learn about the temperature variation curve through the display device 130. Accordingly, medical professionals can take into consideration of how to adjust the length the ablation time in the actual ablation operation so as to prevent excessive ablation or insufficient ablation from happening.

FIG. 8 is a flowchart illustrating steps of an electromagnetic thermotherapy estimation method in an embodiment of the invention. Referring to FIG. 1 and FIG. 8, the method of the present embodiment is at least applicable to the electromagnetic thermotherapy estimation system in FIG. 1. In the present embodiment, the electromagnetic thermotherapy estimation system 100 is adapted to estimate the ablation result of the biological tissue ablated by the needle heated by the electromagnetic coil. In light of the above, the electromagnetic thermotherapy estimation method of the present embodiment can include the following steps. First of all, in step S810, the electromagnetic thermotherapy estimation system 100 receives a plurality of setting parameters through the input device 110, and the setting parameters include a needle tip depth and a current magnitude. Next, in step S820, the processing device 120 of the electromagnetic thermotherapy estimation system 100 calculates ablation range data and temperature envelope data corresponding to the needle tip depth and the current magnitude according to an ablation range estimation model, a temperature envelope estimation model and the setting parameters. Accordingly, the electromagnetic thermotherapy estimation method of the present embodiment may be used to estimate and simulate the ablation range data and the temperature envelope data related to the electromagnetic thermotherapy according to the setting parameters inputted by medical professionals.

In addition, the ablation range data and the temperature envelope data obtained by using the electromagnetic thermotherapy estimation method of the present embodiment may be presented by the display device 130, but the invention is not limited thereto. In one embodiment, the electromagnetic thermotherapy estimation system 100 may also output the estimated data in each of the foregoing embodiments by other means, which are not particularly limited in the invention. In addition, sufficient teaching, suggestion, and implementation illustration regarding the electromagnetic thermotherapy estimation method of the present embodiment may be obtained from the foregoing embodiments depicted in FIG. 1 to FIG. 7, and thus related description thereof are not repeated hereinafter.

In summary, the electromagnetic thermotherapy estimation system and the electromagnetic thermotherapy estimation method according to the embodiments of the invention can be used to calculate the corresponding ablation range data and the temperature envelope data according to the ablation range estimation model and the temperature envelope estimation model created in advance in the database together with the inputted needle tip depth and the current magnitude. Further, the corresponding temperature variation data may also be calculated according to the temperature characteristic equation created in advance in the database and the inputted ablation time. In this way, the electromagnetic thermotherapy estimation system can display the ablation range, the temperature envelope and the temperature variation curve related to the ablated biological tissue according to the ablation range data, the temperature envelope data and the temperature variation data through the display device. As a result, the electromagnetic thermotherapy estimation system and the electromagnetic thermotherapy estimation method according to the embodiments of the invention can effectively provide accurate preoperative estimation information for medical professionals so as to effectively improve the ablation safety and the thermotherapy quality.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An electromagnetic thermotherapy estimation system adapted to estimate an ablation result of a biological tissue ablated by a needle induced by an electromagnetic coil, wherein the electromagnetic thermotherapy estimation system comprises:
an input device, configured to receive a plurality of setting parameters, the setting parameters comprising a needle tip depth and a current magnitude; and
a processing device, electrically connected to the input device, and having a database configured to store an ablation range estimation model and a temperature envelope estimation model,
wherein the processing device is configured to calculate ablation range data and temperature envelope data corresponding to the setting parameters according to the ablation range estimation model, the temperature envelope estimation model, and the setting parameters.

2. The electromagnetic thermotherapy estimation system according to claim 1, wherein the database is further configured to store a plurality of first thermal physical parameters of the biological tissue and store a plurality of second thermal physical parameters of the needle and the electromagnetic coil,
wherein the processing device is configured to perform an electromagnetic simulation analysis and a heat transfer simulation analysis according to the first thermal physical parameters and the second thermal physical parameters so as to create the ablation range estimation model and the temperature envelope estimation model.

3. The electromagnetic thermotherapy estimation system according to claim 2, wherein the first thermal physical parameters comprise a density, a specific heat capacity value, a thermal conductivity and an electrical conductivity of the biological tissue.

4. The electromagnetic thermotherapy estimation system according to claim 2, wherein the second thermal physical parameters comprise a density, a specific heat capacity value, a thermal conductivity and an electrical conductivity of the needle and a surface current density, a radiant flux and an electrical conductivity of the electromagnetic coil.

5. The electromagnetic thermotherapy estimation system according to claim 1, wherein the setting parameters further comprise an ablation time, and the processing device calculates temperature variation data of the needle according to a temperature characteristic equation and the setting parameters.

6. The electromagnetic thermotherapy estimation system according to claim 5, wherein the temperature characteristic equation comprises a plurality of parameter values, and the parameter values comprise a needle tip temperature, a needle tail temperature, a nonlinear regression parameter value, a thermotherapy execution time and a time-varying parameter value of a temperature index.

7. The electromagnetic thermotherapy estimation system according to claim 5, wherein the processing device performs a plurality of sampling ablation operations having different ablation times, different needle tip depths and different current magnitudes on another biological tissue in advance by the needle, and analyzes a needle tip temperature and a needle tail temperature of the needle in each of the sampling ablation operations through a nonlinear regression analysis so as to create the temperature characteristic equation.

8. The electromagnetic thermotherapy estimation system according to claim 7, further comprising:
a driving circuit, electrically connected to the electromagnetic coil, and configured to generate an alternating magnetic field through the electromagnetic coil so as to induce the needle;
a current sensor, electrically connected to the driving circuit, and configured to sense a plurality of current values provided by the driving circuit respectively corresponding to a plurality of sampling ablation results; and
a temperature sensor, electrically connected to the needle, and configured to sense a plurality of temperature values of the needle respectively corresponding to the sampling ablation results,
wherein the processing device creates the temperature characteristic equation through the nonlinear regression analysis according to the current values and the temperature values corresponding to the different ablation times.

9. The electromagnetic thermotherapy estimation system according to claim 5, further comprising:
a display device, electrically connected to the processing device, and configured to display an ablation range image and at least one temperature envelope corresponding to the setting parameters according to the ablation range data and the temperature envelope data,
wherein the ablation range image comprises marking a maximum ablation width.

10. The electromagnetic thermotherapy estimation system according to claim 9, wherein the display device is further configured to display a temperature variation curve of the needle according to the temperature variation data.

11. An electromagnetic thermotherapy estimation method adapted to estimate an ablation result of a biological tissue ablated by a needle induced by an electromagnetic coil, wherein the electromagnetic thermotherapy estimation method comprises:
receiving a plurality of setting parameters, the setting parameters comprising a needle tip depth and a current magnitude; and
calculating ablation range data and temperature envelope data corresponding to the setting parameters according to an ablation range estimation model, a temperature envelope estimation model and the setting parameters.

12. The electromagnetic thermotherapy estimation method according to claim 11, further comprising:
performing an electromagnetic simulation analysis and a heat transfer simulation analysis according to a plurality of first thermal physical parameters of the biological tissue and a plurality of second thermal physical parameters of the needle and the electromagnetic coil so as to create the ablation range estimation model and the temperature envelope estimation model.

13. The electromagnetic thermotherapy estimation method according to claim 12, wherein the first thermal physical parameters comprise a density, a specific heat capacity value, a thermal conductivity and an electrical conductivity of the biological tissue.

14. The electromagnetic thermotherapy estimation method according to claim 12, wherein the second thermal physical parameters comprise a density, a specific heat capacity value, a thermal conductivity and an electrical conductivity of the needle and a surface current density, a radiant flux and an electrical conductivity of the electromagnetic coil.

15. The electromagnetic thermotherapy estimation method according to claim 11, wherein the setting parameters further comprise an ablation time, and the electromagnetic thermotherapy estimation method further comprises:
calculating temperature variation data of the needle according to a temperature characteristic equation and the setting parameters.

16. The electromagnetic thermotherapy estimation method according to claim 15, wherein the temperature characteristic equation comprises a plurality of parameter values, and the parameter values comprise a needle tip temperature, a needle tail temperature, a nonlinear regression parameter value, a thermotherapy execution time and a time-varying parameter value of a temperature index.

17. The electromagnetic thermotherapy estimation method according to claim 15, further comprising:
performing a plurality of sampling ablation operations having different ablation times, different needle tip depths and different current magnitudes on another biological tissue in advance by the needle; and
analyzing a needle tip temperature and a needle tail temperature of the needle in each of the sampling ablation operations through a nonlinear regression analysis so as to create the temperature characteristic equation.

18. The electromagnetic thermotherapy estimation method according to claim 17, wherein the step of creating the temperature characteristic equation comprises:
generating an alternating magnetic field by driving the electromagnetic coil through a driving circuit so as to induce the needle;
sensing a plurality of current values provided by the driving circuit respectively corresponding to a plurality of sampling ablation results through a current sensor;
sensing a plurality of temperature values of the needle respectively corresponding to the sampling ablation results through a temperature sensor; and
creating the temperature characteristic equation through the nonlinear regression analysis according to the current values and the temperature values corresponding to the different ablation times and the different needle tip depths.

19. The electromagnetic thermotherapy estimation method according to claim 15, further comprising:
displaying an ablation range image and at least one temperature envelope corresponding to the setting parameters through a display device according to the ablation range data and the temperature envelope data, wherein the ablation range image comprises marking a maximum ablation width.

20. The electromagnetic thermotherapy estimation method according to claim 19, further comprising:
displaying a temperature variation curve corresponding to the setting parameters through the display device according to the temperature variation data.
